(19) United States
(12) Patent Application Publication
(10) Pub. No.: US 2023/0069771 A1
(43) Pub. Date: Mar. 2, 2023

(54) DRIVE UNIT FOR INTRAVASCULAR CIRCULATORY SUPPORT SYSTEMS

(71) Applicant: NuPulseCV, Inc., Raleigh, NC (US)

(72) Inventor: Douglas Edward Ivers, Cary, NC (US)

(21) Appl. No.: 17/878,632

(22) Filed: Aug. 1, 2022

Related U.S. Application Data
(60) Provisional application No. 63/259,918, filed on Aug. 2, 2021.

(51) Int. Cl. A61M 60/497; A61M 60/139; A61M 60/295; A61M 60/515

(52) U.S. Cl. CPC A61M 60/497; A61M 60/139; A61M 60/295; A61M 60/515

(57) ABSTRACT

A drive unit for intravascular circulatory support systems may include a motor, a ball nut, a ball screw, and a bellows. The motor may include a rotor and a stator. The ball nut may be affixed to the rotor. The bellows may have a first end and an second end and a bellows cavity located there between. The first end may be in fixed position and the second end may be defined by a dynamic flange having a recess carried by the bellows cavity. In turn, the recess of the dynamic flange may carry at least a portion of the motor. The second end may also receive the ball screw. Rotation of the rotor causes linear motion of the ball screw within the ball nut to actuate the bellows.

### TECHNICAL FIELD

The present technology is directed to systems, devices, and methods for treating heart failure, and in particular to a drive unit for intravascular circulatory support systems.

### BACKGROUND

The prevalence of heart failure is increasing worldwide and is an expensive burden on health care providers. Despite advances in medical care, prognosis for patients with heart failure remains poor, especially in patients having advanced stage heart failure. This is due in part to limited therapy options for such patients. Counterpulsation using an intravascular circular support system is a treatment option for heart failure. Such a system may include a thin, flexible tube called a catheter with a long, thin balloon attached to the catheter tip. The balloon is positioned in the aorta of a patient. The other end of the catheter may attach to a computer console or “drive unit” with a mechanism for inflating and deflating the balloon at the proper time during the heartbeat. The balloon operational acts as a balloon pump (i.e., an intra-aortic balloon pump (IABP)). The drive unit may inflate the balloon when the heart relaxes to push blood towards the end-organs and the coronary arteries to perfuse the heart. And before the left ventricle contracts, the drive unit may cause the balloon to deflate, reducing the pressure that the heart has to pump against. This enables the heart to pump more blood into the body while using less energy. The drive unit may continue to inflate and deflate the balloon in sync with the heartbeat.

Conventional drive units may employ helium to inflate and deflate the balloon. However, there are technical costs, patient usability and portability issues, and safety risks associated with using helium. For instance, the drive unit must contain (or be connected to) a storage tank containing helium. As a result, conventional drive units are too large and heavy for portability. Typical drive units that use helium weigh 50-100 lbs and are confined to in-hospital use. Further, in the event of a balloon leak or rupture, helium is not readily absorbed in blood and poses a significant thrombogenic and stroke risk to the patient.

Further, balloon inflation and deflation that is poorly synchronized with the patient’s heartbeat can be massively detrimental, or at least therapeutically unproductive. For example, early balloon inflation can result in increased afterload on the left ventricle (i.e., the amount of resistance the heart must overcome to open the aortic valve and push the blood volume out into the systemic circulation). When the balloon inflates too early, the left ventricle can still be in the process of contracting, trying to eject blood through the open aortic valve. Thus, the left ventricle might be working against not only the systemic vascular resistance, but also the additional resistance caused by the inflated balloon in the aorta, which is obstructive to blood flow from the heart. Also, some blood can be pushed backwards through the aorta and into the left ventricle, increasing ventricular volume and putting greater stress on its walls. Late balloon inflation can result in decreased diastolic augmentation. To be therapeutically effective, the drive unit might inflate the balloon just after the aortic valve closes. If inflation occurs much later after the aortic valve closure, the balloon does not have adequate time to push blood to the body and the heart during diastole, reducing therapeutic efficacy. Early balloon deflation does not reduce ventricular workload and myocardial oxygen demand as effectively. When the balloon deflates too early, the aortic pressure may have had time to equalize, diastolic pressure near the heart may revert to its unassisted level, and there may be no reduction in the duration of left ventricular isovolumetric contraction or the afterload that the heart may have to eject against. The result of early balloon deflation may be a failure to decrease myocardial oxygen demand. While even early balloon deflation may offer some diastolic augmentation benefit, the left ventricle may not be assisted in opening the aortic valve, and so there is reduced afterload reduction. Late balloon deflation may increase afterload because the aortic end-diastolic pressure does not have enough time to decrease by the time the left ventricle is ready to contract again.

The drive unit may cause the balloon to inflate and deflate at the correct time based, at least in part, on real-time sensing and analysis of a patient’s electrocardiogram (ECG or EKG). However, even with perfect signal sensing and delivery to the drive unit, the mechanical inefficiencies of the drive unit may cause some delay in actual inflation and deflation cycles of the balloon. For some cardiovascular conditions, this delay may prevent therapeutic application of counterpulsation using an IABP. Heart arrhythmias, such as atrial fibrillation, are especially sensitive to such delays because of the irregular and often rapid heart rate that occurs when the two upper chambers of the heart experience chaotic electrical signals. For example, to ensure that the inflated balloon is not obstructive in patients with a-fib or other arrhythmias, the balloon should be deflated by at least 50% within approximately one hundred milliseconds of receipt of an R-wave in a QRS complex. Thus, there is a need for a drive unit for an intravascular circulatory support system (e.g., one include an IABP) that is fast enough to provide a safe and effective therapy for patients suffering from heart arrhythmias, irregular heartbeats, or a-fib. There is another need for a drive unit that does not require expensive, large, heavy, and therefore largely non-portable helium tanks. There is similarly a need for such a drive unit that uses a fluid other than helium to avoid or mitigate the risk presented by using helium to inflate a balloon. Finally, there is a need for such a drive unit with improved portability with decreased weight and size compared to conventional drive units.

### BRIEF DESCRIPTION OF THE DRAWINGS

Many aspects of the present technology can be better understood with reference to the following drawings. The components in the drawings are not necessarily to scale, and instead emphasis is placed on illustrating clearly the principles of the present disclosure.

FIG. 1 illustrates an example of an intravascular circulatory support system implanted within a patient’s vasculature.

FIG. 2A illustrates a top perspective external view of an exemplary first embodiment of a drive unit for an intravascular circulatory support system.

FIG. 2B illustrates a top external view of the drive unit of FIG. 2A.

FIG. 2C illustrates a side external view of the drive unit of FIG. 2A.

FIG. 2D illustrates an end external view of the drive unit Of FIG. 2A.

FIG. 2E illustrates a cross-sectional view of the drive unit of FIG. 2A along section A-A of FIGS. 2 A and 2D.

FIG. 2F illustrates an exploded view of a portion of the drive unit of FIG. 2A along section A-A of FIGS. 2A and 2D.

FIG. 3A illustrates a top external perspective view of an exemplary second embodiment of a drive unit for an intravascular circulatory support system.

FIG. 3B illustrates an external end view of the drive unit of FIG. 3A.

FIG. 3C illustrates a first side external view of the drive unit of FIG. 3A.

FIG. 3D illustrates a second side external view of the drive unit of FIG. 3A.

FIG. 3E illustrates a cross-sectional view of the drive unit of FIG. 3A along section C-C of FIGS. 3C and 3D.

FIG. 4 illustrates a block diagram of certain control components applicable to the drive units of FIGS. 2A and 3A.

### DETAILED DESCRIPTION

Specific details of several embodiments of the technology are described below with reference to the figures. Although many of the embodiments are described below with respect to use of intravascular circulatory support systems/intravascular ventricular assist devices (“iVAD”) that position an IABP/balloon in the aorta to provide counterpulsation that helps move blood through the body, other applications and other embodiments in addition to those described herein are within the scope of the technology. For example, several other embodiments of the technology can have different configurations, components, or procedures than those described herein, and features of the embodiments shown can be combined with one another. A person of ordinary skill in the art, therefore, will accordingly understand that the technology can have other embodiments with additional elements, or the technology can have other embodiments without several of the features depicted and described below.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

### Heart Failure and Circulatory Support Systems

Heart failure occurs when the heart is unable to maintain blood flow to meet the body’s needs. This can occur if the heart cannot pump or fill adequately during contraction and relaxation, respectively. Heart failure is a common, costly, and potentially fatal condition. For example, heart failure currently affects about 6.5 million patients in the United States and is expected to increase by 46% by 2030. The stage/severity of heart failure can be defined using New York Heart Association (NYHA) classes, with NYHA Class I representing an early stage disease and NYHA Class IV representing a late stage disease. Current treatment options for heart failure depend on the stage of heart failure, and include, among other options, pharmacological therapy, cardiac resynchronization therapy (CRT), long-term mechanical circulatory support (e.g., left ventricular assist devices, or “LVAD”), and heart transplantation. Pharmacological therapy and CRT are typically used in relatively early stage cases (e.g., in patients with NYHA Class II or early NYHA Class III heart failure). However, these therapies typically only delay the progression of heart failure, meaning that even patients who initially respond to pharmacological therapy or CRT typically experience disease progression and required more advanced therapeutic interventions.

Prognosis for patients with heart failure remains poor: one-year mortality is about 15.0% for NYHA Class III patients and about 28.0% for NYHA Class 4 patients. This is at least partially due to the limited number of treatment options available for patients with late NYHA Class III/early NYHA Class IV heart failure. For example, while heart transplantation offers the best opportunity for long-term survival in late NYHA Class IV patients, this option is limited due to the scarcity of donor organs (e.g., approximately 2000 per year in the US, 200 per year in Canada, and less than 100 per year in Japan). Accordingly, many NYHA Class III and Class IV patients must rely on other treatments. For example, some patients receive LVAD therapy as a bridge to heart transplant or as a standalone therapy. However, LVAD therapy has several inherent shortcomings that limit their widespread use. Current LVAD therapies are expensive (e.g., over $100,000), typically require a major surgical procedure to implant (typically a sternotomy or thoracotomy), typically require use of cardiopulmonary bypass during the implant procedure, and typically require blood products (e.g., about 11.6 units of blood products). LVAD therapies that are implanted through less-invasive means (e.g., percutaneously) are only used for short-term circulatory support. Furthermore, postoperative care of patients who receive a LVAD can be challenging and costly, and patient anxiety can be high because the devices cannot be shut off or lose power for more than a few minutes. LVAD therapy is also associated with several serious adverse events such as device failure, thrombosis, thromboembolism, stroke, infection, and bleeding. For at least these reasons, LVAD therapy is typically reserved for patients with end-stage heart failure who have limited options (e.g., late NYHA Class IV). This leaves a large percentage of heart failure patients who have cases that are too advanced for CRT but are not yet severe enough to justify LVAD therapy/heart transplantation (e.g., late NYHA Class III/early NYHA Class IV patients) without effective treatment options. There are currently about 1.6 million patients in the United States and about 3.9 million patients in Europe with late Class III/early Class IV heart failure, representing a large patient population with limited treatment options.

Another treatment option for heart failure is counterpulsation therapy using an intraaortic balloon pump (IABP). Counterpulsation therapy is achieved by rapidly inflating a balloon positioned in the patient’s aorta immediately after aortic valve closure (dicrotic notch) and rapidly deflating the balloon just before the onset of systole. The rapid inflation of the balloon increases the diastolic aortic pressure by 25-70%, augmenting end-organ and coronary perfusion. The rapid deflation of the balloon reduces the ejection pressure of the native ventricle, reducing afterload and left ventricular external work.

Counterpulsation therapy is an attractive therapy option because using an IABP is much simpler than implanting and using an LVAD and is associated with fewer adverse events. For example, a physician can implant an IABP without directly cannulating the heart. However, conventional counterpulsation systems implanted through minimally invasive procedures can only be used for short durations. This is this case for several reasons. For example, the arterial access (e.g., the femoral artery), the durability of the IABP, and biocompatibility issues can limit the use of IABP to short durations of less than about 14 days. Longer duration of IABP support (>14 days) can lead to an increase in the frequency of vascular complications, infections, and bleeding. Moreover, in its current form, a catheter mounted IABP advanced retrograde from the femoral artery into the descending aorta requires the patient to remain supine for the duration of therapy. Consequently, patients cannot be ambulatory or be discharged from the hospital. These limitations prevent the IABP from being used as an extended therapy for heart failure and instead are used in short term settings, such as in patients awaiting transplant and in patients undergoing coronary artery bypass surgery.

The assignee/applicant (NuPulseCV, Inc.) has developed various counterpulsation support systems designed to provide longer-term support to patients suffering from heart failure as compared to the above-described conventional systems. Such improved counterpulsation support systems are described in U.S. Pat. No. 7,892,162 entitled “ARTERIAL INTERFACE” filed Oct. 22, 2009, U.S. Pat. No. 8,066,628 entitled “INTRA-AORTIC BALLOON PUMP AND DRIVER” filed Oct. 22, 2010, U.S. Pat. Application Serial No. 15/685,553 entitled “BLOOD PUMP ASSEMBLY AND METHOD OF USE THEREOF” filed Aug. 24, 2017, and U.S. Pat. Application Serial No. 16/876,110 entitled “INTRAVASCULARLY DELIVERED BLOOD PUMPS AND ASSOCIATED DEVICES, SYSTEMS, AND METHODS” filed on May 17, 2020, the entire disclosures of each of the foregoing is incorporated by reference herein.

The IABPs described in the incorporated disclosures offer long-term or chronic counterpulsation therapy for heart failure patients with components that, at least in certain instances, can be implanted using minimally invasive percutaneous procedures. For example, the IABP can be implanted minimally invasively without entering the chest, and generally do not require cardiopulmonary bypass or administration of blood products.

And, the present technology offers a drive unit that can direct a fluid (gas or liquid, e.g., air) into an internal volume of an balloon or expandable member (e.g., the IABPs described in the above-incorporated disclosures) that has been implanted into the patient’s vasculature while minimizing the mechanical inefficiencies that may cause mismatching the balloon inflation and deflation cycles with a patient’s EKG, even in cases of arrhythmia such as atrial fibrillation, as described in greater detail below.

### Select Embodiments of Chronic Intravascular Circulatory Support Systems

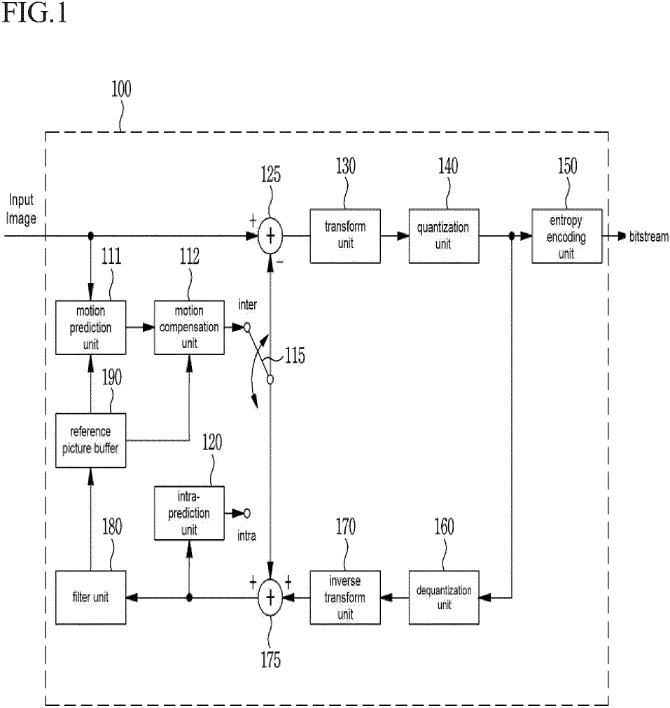

FIG. 1 illustrates a circulatory support and/or intravascular ventricular assist system 100 configured in accordance with select embodiments of the present technology. The system 100 can include an expandable member 110 implantable in an aorta of a patient suffering from heart failure. Expandable member 110 may be referred to as an IABP or balloon. The system 100 can further include a first pneumatic driveline 120 (also referred to as an “internal driveline”), an arterial interface device or stopper 130, a second pneumatic driveline 140, a skin interface device 190, a drive unit 150, and sensors 160. When implanted, the system 100 can provide counterpulsation therapy to a patient suffering from heart failure.

The expandable member 110 can be a balloon or other element that can change size and/or shape in response to being filled with a gas or liquid. For example, in some embodiments the expandable member 110 is a balloon composed of a biocompatible, non-thrombogenic elastomeric material (e.g., Biospan®-S). The expandable member 110 can also be made of other suitable materials. The expandable member 110 is transitionable between at least a first state in which it is generally deflated and a second state in which it is generally inflated. The expandable member 110 has a first volume when in the first (e.g., deflated) state and a second volume that is greater than the first volume when in the second (e.g., inflated) state.

Accordingly, the expandable member 110 can provide counterpulsation therapy by repeatedly transitioning between the first state and the second state. To transition the expandable member 110 between the first state and the second state, the drive unit 150 can direct a fluid (gas or liquid, e.g., air) into an internal volume of the expandable member 110 via the first pneumatic driveline 120 and the second pneumatic driveline 140, as described in greater detail below. The expandable member 110 can also be sized and/or shaped to reduce and/or prevent the expandable member 110 from blocking arteries branching from the aorta, such as the renal arteries. In some embodiments, the expandable member 110 may also have certain features generally similar to those described in disclosures incorporated herein by reference. In some embodiments, the expandable member 110 includes an expandable end effector other than or in addition to a balloon.

The drive unit 150 can generate gas flow into and out of the expandable member 110 via the first pneumatic driveline 120 and the second pneumatic driveline 140 that converge to form an external pneumatic driveline 172 extending from the drive unit 150. For example, the drive unit 150 can generate a positive pressure to accelerate gases into the expandable member 110 via the first pneumatic driveline 120 and the second pneumatic driveline 140, thereby inflating the expandable member 110. The drive unit 150 can also induce a negative pressure to withdraw gases from the expandable member 110 via the first pneumatic driveline 120 and the second pneumatic driveline 140, thereby deflating the expandable member 110. The drive unit 150 can induce gas flow into and out of the expandable member 110 through a bellows (not depicted in FIG. 1). In some embodiments, the drive unit 150 can control the volume of air being pushed into the expandable member 110 to avoid overinflating the expandable member 110. For example, in an embodiment utilizing a bellows to generate air flow, the volume of airflow generated by the bellows (e.g., the volume of the bellows) can correspond to an interior volume of the expandable member 110. One of skill in the art will recognize that the volume of the bellows may not be identical to the interior volume of the balloon due to changes in relative pressure. In some embodiments, the drive unit 150 uses ambient air from the environment surrounding the drive unit 150 (e.g., “room air”) to drive operation of the system 100. Using ambient air is expected to reduce the size, weight, and/or cost of the drive unit 150 relative to a drive unit that relies on an internal gas or fluid supply (e.g., helium tanks). For example, in some embodiments the drive unit 150 can weigh about 3 kg or less. Also, in case of a balloon leak or rupture, air is more readily absorbed in blood and poses a lower thrombogenic and stroke risk compared to helium. The driveline can be disconnected near the skin interface device 190 when the system 100 is not being actively used.

The system 100 may allow chronic support of heart function and blood flow, while still allowing the patient to remain ambulatory. Typical ventricular assist devices require femoral access for the driveline and/or connection to large, stationary external control units, and therefore confine the patient to a hospital bed in the supine position for the duration of therapy. In contrast, the system 100 allows the patient to move about relatively unencumbered. Moreover, the therapy level provided by the system 100 can be adjusted to match patient need. For example, the volume displacement and support ratio (e.g., 1:1, 1:2, 1:3 support) provided by the expandable member 110 can be adjusted for each patient to vary the support provided. One of skill in the art will recognize that the support ratio measures the ratio of beats to inflations of the balloon. For example a 1:1 support ratio indicates the for every beat there is a corresponding inflation of the balloon, whereas a 1:2 support ratio indicates that there are two beats before each inflation, and a 1:3 support ratio indicates that there are three beats before each inflation. The drive unit 150 can have a user interface (not depicted) where a user can control the volume displacement or support ratio. Alternatively, drive unit 150 can be controlled via other external inputs (e.g., using a corresponding tablet or other device that is in electrical communication with drive unit 150). Gradually reducing the volume displacement over time (e.g., by changing the desired travel of an internal bellows mechanism as a percent of the full travel) can result in a controlled loading of the heart which in some instances may be beneficial for cardiac recovery. Furthermore, unlike conventional circulatory support systems, the system 100 can be turned off to, for example, assess the ability of the patient to handle cardiac demand without support before removing the system 100, or for another suitable reason. In some embodiments, for example, the expandable member 110 is designed to remain in the aorta in a deflated condition for a relatively prolonged duration (e.g., for 23 hours in a single day). This is in direct contrast to conventional devices, which often must be removed if turned off for more than about fifteen minutes because reactivation could result in a shower of emboli that formed when in the inactive state.

### Drive Unit

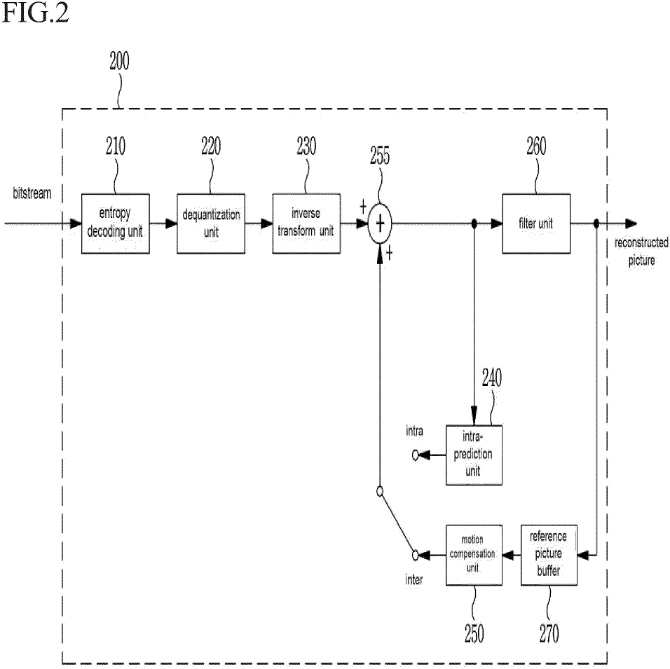

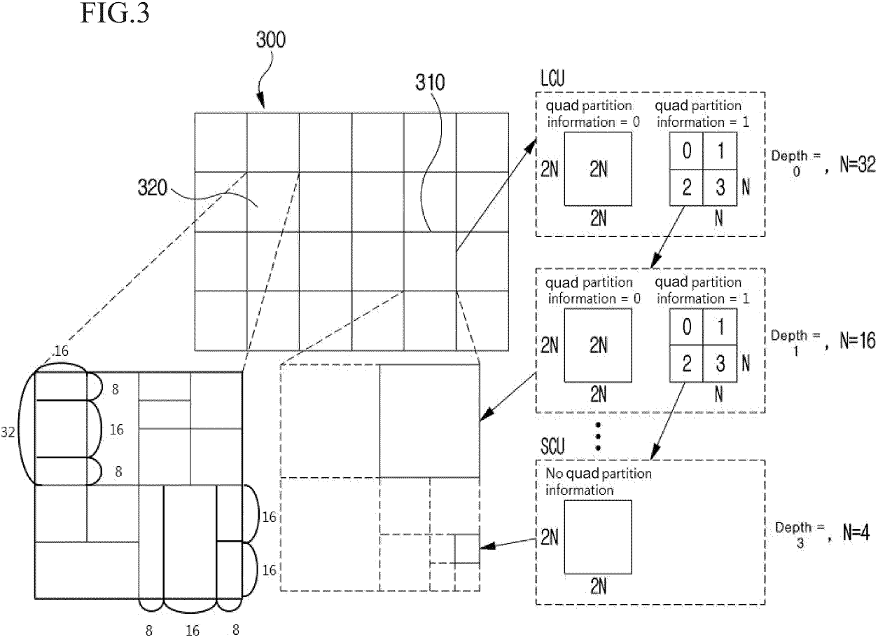

The present disclosure contemplates at least two embodiments of drive unit 150. The first embodiment is depicted in FIGS. 2A-2F and refers to the drive unit by reference numeral 200. The second embodiment is depicted in FIGS. 3A-3E and refers to the drive unit by reference numbers 300.

FIG. 2A depicts a first embodiment of drive unit 200. Drive unit 200 may include an outer case 202 having various ports 204. Ports 204 may be used for, among other things, display readouts, power buttons, ventilation, and connectivity to other system components (e.g., pneumatic driveline 172).

With reference to FIGS. 2B-2D, the drive unit 200 may outer case 202 having top access plate 208 affixed using fasteners 206. With reference to FIGS. 2E-2F, the drive unit 200 may include a bellows 220 and a motor 222 (e.g., an electric motor such as a brushless DC motor. The space to the left of bellows 220 in FIG. 2E may be at least partially occupied by electrical components (not illustrated) used to control motor 222. Such components may include one or more computer processors and memory containing computer processor readable instructions capable of being executed by the computer processor. The same space may be at least partially occupied by mechanical components such as value manifolds (e.g., for coupling bellows output/pneumatic output 223 to an applicable port 204).

Bellows 220 may be an axial expansion bellows that is able to expand and contract along the B-B axis depicted in FIG. 2E in response to rotation of the motor 222 in different directions. In the embodiment depicted by FIGS. 2E and 2F, the bellows 220 is expanded such that the expandable member 110 (FIG. 1) is deflated. When the bellows 220 is compressed by motor 222, the expandable member 110 (FIG. 1) is inflated. The bellows 220 may have a constant cross-sectional geometry along its length so that the volume within the bellows 220 is varied according to the bellows length.

The motor 222 may include a rotor 222a and stator 222b. The rotor 222a may be joined to a rotary-to-linear transformer. For example, the rotor 222a may be joined to a ball nut 224 carrying a ball screw 226 that is affixed to a dynamic flange 228. The bellows 220 may be sealed by the dynamic flange 228 at a first bellows end proximal to the ball screw 226 and by a static flange 229 (FIG. 3E) at a second bellows end that is distal to the ball screw 226. A bellows outlet 233 (FIG. 3E) may allow communication of fluid within the external drive line 172 to the expandable member 110 in response to movement of the bellows 220.

In some embodiments, the bellows outlet 233 may be formed within the static flange 229. The ball screw 226 in combination with the ball nut 224 may form a mechanical rotary-to-linear transformer that converts rotational motion of the motor 222 to linear motion with little friction. Rotation of the ball nut 224 by the rotor 222a within the stationary stator 222b may cause the ball screw 226 to move linearly along axis B-B and, correspondingly, cause linear movement of the bellows 220.

The ball screw 226 may be threaded to provide a helical raceway 226a for balls (not depicted) of the ball nut 224 and may act as a precision screw. Several dimensions may define the ball screw 226 and raceway 226a. For example, the ball screw 226 may include a pitch measuring the distance between grooves of the helical raceway 226a.

The rotor 222a and ball nut 224 assembly may be mounted to a housing 230 of the motor 222 via radial bearings 232. The inner race of the radial bearings 232 may be affixed to the rotor 222a and ball nut 224 assembly while the outer race of the radial bearings 232 and the stator 222b may be affixed to a motor housing 230. Actuation of the motor 222 may cause rotational movement of the rotor 222a and ball nut 224 which may cause balls (not depicted) of the ball nut 224 to ride within the helical raceway 226a of the ball screw 226 and convert the rotational movement of the rotor 222a into linear movement of the ball screw 226 along axis B-B (FIG. 2E). Movement of the ball screw 226 is translated to the bellows 220 via the dynamic flange 228, causing the bellows 220 to expand or contract to create negative or positive fluid flow, respectively, within the external drive line 172, which is in fluid connection with the expandable member 110 via the bellows outlet 233 (FIG. 2E). In some embodiments, the ball screw 226 may be fixedly mated to the dynamic flange 228 via a threaded interface (as depicted in FIG. 2F). In some embodiments, the dynamic flange 228 and ball screw 228 are one continuous piece. When the ball screw 226 is fixedly mated to the dynamic flange 228 or when the ball screw 226 and dynamic flange 228 are one continuous piece, ball screw 228 may directly and efficiently translate its motion to the bellows (e.g., withdrawal of the ball screw 226 away from the static flange 229 may pull the dynamic flange 228 to expand the bellows 220).

With reference to FIGS. 2E-F, and 4, drive unit 200 may include processor 402, memory 404, and drive unit 402. Processor 402 may include one or more dedicated or non-dedicated micro-processors, micro-controllers, sequencers, micro-sequencers, digital signal processors, processing engines, hardware accelerators, applications specific circuits (ASICs), state machines, programmable logic arrays, any integrated circuit(s), discrete circuit(s), etc. that is/are capable of processing data or information, or any suitable combination(s) thereof. Memory 404 may include any suitable non-volatile memory device, chip, or storage device capable such as one or more of: system memory, frame buffer memory, flash memory, random access memory (RAM), read only memory (ROM), a register, and a latch. Processor 402 is capable of executing executable instructions (e.g., as stored in memory 404). Processor 402 may be configured to control motor 222, and by extension, the bellows 220.

For example, an encoder disk 234 and encoder sensor 236 may determine the angular position, speed, and/or direction of the rotor and provide such information as positional feedback signal(s) to a processor 402 and/or memory 404. In other embodiments, a linear position sensor (not depicted) may be used to determine the position of the ball screw 226 or bellows 220 and to provide such positional feedback signal(s). Processor 402 and/or memory 404 may also receive control information from drive unit Ul 406 and provide display information (e.g., status information) to drive unit Ul 406. Relatedly, processor 402 and/or memory 404 may similarly receive EKG signal(s) from skin interface device 190 and one or more sensors 160 (FIG. 1), one or more external control signals (e.g., from a tablet or other computing device (not depicted)), and one or more sensor signals. For example, processor 402 and/or memory 404 may receive a pressure signal as observed by a pressure sensor (not depicted) located in proximity to balloon 110 when disposed in an artery (e.g., the descending aorta, as depicted in FIG. 1) of a patient undergoing therapy (e.g., counterpulsation therapy). The one or more pressure signals may be, indicative of the pressure exerted on balloon 110 in such artery and/or the pressure within such artery.

Processor 402 may use one or more of the positional feedback signals, drive unit Ul 406 control signals, EKG signals, external control signals, and sensor signals to control motor 222. For example, EKG signals may be used to ensure proper timing of the inflation and/or deflation of the balloon 110 (e.g., to pursue counterpulsation). And drive unit Ul 406 control signal and external control signals may be used to change the volume displacement and/or support ratio, as discussed supra.

Other embodiments may employ linear brushless DC motors, solenoids, and/or piezo electric actuators to compress and expand bellows 220.

Drive unit 200 may be used to effectively inflate and deflate inflatable member 110 using ambient air to provide counterpulsation therapy in patients with heart failure. The following component values set forth in the table below may be efficient and/or economical for such purposes. The two columns of numbers represent suitable dimensions for counterpulsation using the drive unit 200 and an approximately 20-60 cc balloon as inflatable member 110.

| Drive Unit 200 Component (units) | Ex 1. Dimensions | Ex. 2 Dimensions |
|---|---|---|
| Bellows Outside Diameter (mm) | 90 | 76.2 |
| Bellows Piston Area (cm2) | 48.2 | 29.5 |
| Bellows Travel (mm) | 17.7 | 29 |
| Drive Unit Height (mm) | 87 | 128 |
| Envelope Volume / Size of Bellows Plus Drivetrain (cc) | 557 | 584 |

Although effective for counterpulsation in general, drive unit 200 as configured above, uses a significant amount of power merely to overcome the rotational inertia of rotor 222a and may not be “fast enough” to treat patients with heart arrhythmias, irregular heartbeats, or a-fib. Ball screws pitch may be selected or adjusted to improve efficiency of the drive unit. In particular, adjusting the ball screw pitch (and/or adjusting the diameter of the bellows 220) may better match the impedance of the motor 222 and the impedance of the pneumatic load on the drive unit 200 (e.g., the collectively pneumatic load of balloon 110, first pneumatic driveline 120, second pneumatic driveline 140, and external driveline 172). Such an improvement in efficiency may improve the battery life of any battery employed to drive motor 222.

With reference to FIG. 3A, a second embodiment of the drive unit 300 may also include an outer case 302 having various ports 304. Ports 304 may be used for, among other things, display readouts, power buttons, ventilation, and connectivity to other system components (e.g., pneumatic driveline 172).

With reference to FIGS. 3B-3D, outer case 302 may include an end access plate 308 securedly fixed thereto. With reference to FIG. 3E, the drive unit 300 may include a bellows 320 and a motor 322 (e.g., an electric motor such as a brushless DC motor). Bellows 320 may be an axial expansion bellows that is able to expand and contract along the D-D axis depicted in FIG. 3E in response to rotation of the motor 322 in different directions. As depicted in FIG. 3E, the bellows 320 may include a dynamic flange 328. The dynamic flange 328 may be sized and shaped to receive or house at least a portion of the motor 322 such that at least a portion of the motor 322 is nested within a bellows cavity 320a. For example, the direction of the recess 328a may be toward the bellows cavity 320 and take a three-dimensional shape (e.g., a cylinder) suitable for receiving or housing and nesting at least a portion of the motor 322 within the bellows cavity 321a while maintaining sufficient stroke length to provide effective air flow within the external drive line 172. The recess 328a may allow a reduction in size and weight of the outer case 302, improving mobility of the system (e.g., as compared to the size of outer case 202). In the embodiment depicted in FIG. 3E, the bellows 320 is depicted in an expanded or uncompressed state such that the expandable member 110 (FIG. 1) is deflated.

The motor 322 includes a rotor 322a and stator 322b. As with the first embodiment 200, the rotor 322a may be shaped to join a rotary-to-linear transformer, for example, a ball nut 324 carrying a ball screw 326 that is affixed to a dynamic flange 328 via a threaded interface. In other embodiments, the rotor 323a and the ball nut 324 may be formed as a single piece. In some embodiments, the ball screw 326 is hollow to reduce weight. The rotor 323a and ball nut 324 assembly may be mounted to a housing of the motor 332 via radial bearings 330. The inner race of the radial bearings 330 may be affixed to the rotor 323a and ball nut 324 assembly while the outer race of the radial bearings 330 and the stator 323b may be affixed to a motor housing 332. Like the first embodiment, actuation of the motor 322 may cause rotational movement of the rotor 323a and ball nut 324 which may translate into linear movement of the ball screw 326 along axis D-D. Movement of the ball screw 326 may be translated to the bellows 320 via the dynamic flange 328, causing the bellows 320 to expand or contract to create negative or positive fluid flow, respectively, within the external drive line 172, which is in fluid connection with the expandable member 110 via a bellows output/pneumatic output 333 associated with the static flange 329. Other embodiments may employ linear brushless DC motors, solenoids, and/or piezo electric actuators to compress and expand bellows 320.

The ball screw 326 may be mated to the dynamic flange 328 (or the two components may be one continuous piece) to allow the ball screw 328 to directly and efficiently translate its motion to the bellows 320 while the rotor 322a and ball nut 324 spin within the stator 322b around axis D-D. Rotation of the rotor 322a may carry the balls of the ball nut 324 within the helical raceway 226a of the ball screw 226, thus causing movement of the ball screw 226 along axis D-D.

In reference to FIGS. 2E-F and 4, an encoder disk 334 and encoder sensor 336 may determine the angular position, speed, and/or direction of the rotor and provide such information to a processor via electrical feedback signals (not depicted). In other embodiments, a linear position sensor (not depicted) may be used to determine the position of the ball screw 326 or bellows 320 and to provide that information to such processor. Such a processor may also receive EKG signals from skin interface device 190 and one or more sensors 160 (FIG. 1), suitable external control signals (e.g., from a tablet (not depicted) configured to control motor 322 and the travel of bellows 220, a user interface operatively coupled to the case 302 (not depicted), etc.), and other sensors (e.g., pressure sensors (not depicted) capable of sensing the pressure in the descending aorta).

Drive unit 300 may be used to effectively inflate and deflate inflatable member 110 using ambient air to provide counterpulsation therapy in patients with heart failure, including patients with heart arrhythmias, irregular heartbeats, or a-fib. The following ranges of component values may be efficient and/or economical for counterpulsation using drive unit 300 and an approximately 20-60 cc balloon as inflatable member 110.

| Drive Unit 300 Component (units) | Range of Dimensions | |
|---|---|---|
| Bellows Outside Diameter (mm) | 125 | 111 |
| Bellows Piston Area (cm2) | 92.3 | 73 |
| Bellows Travel (mm) | 8 | 11.5 |
| Drive Unit Height (mm) | 71 | 75 |
| Envelope Volume / Size of Bellows Plus Drivetrain (cc) | 865 | 726 |
| Ball Screw Pitch (mm) | 3.5 | 5.5 |
| Peak Motor Torque (N*m) | 0.22 | 0.19 |
| Plateau Speed (rpm) | 1380 | 1365 |
| Average Power (watts) | 5.37 | 5.1 |

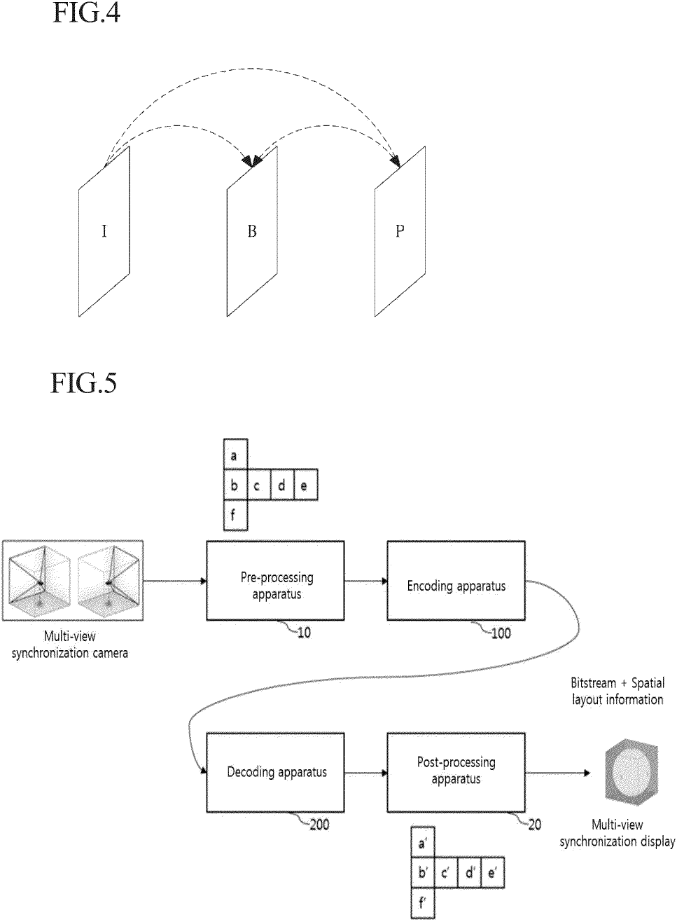

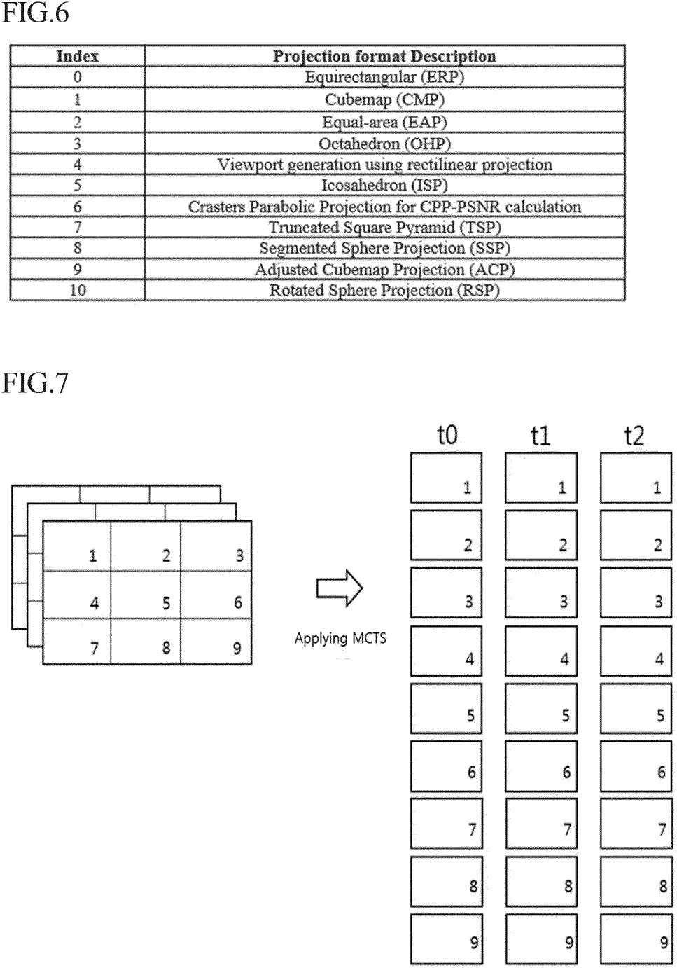

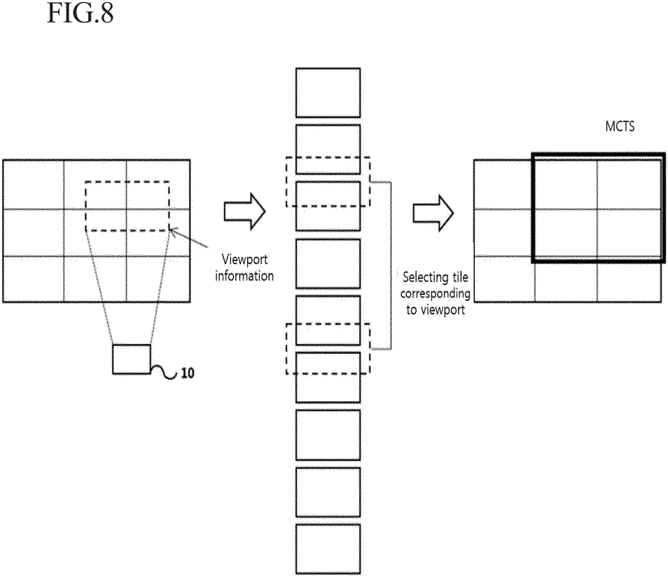

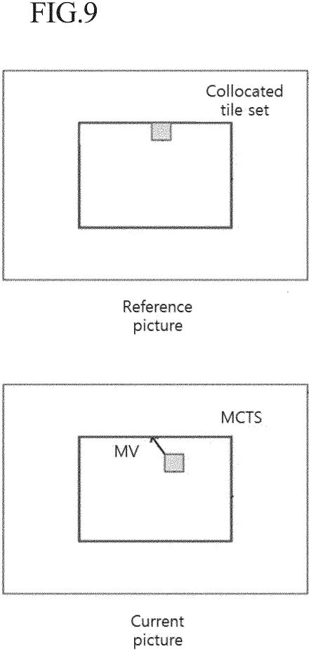

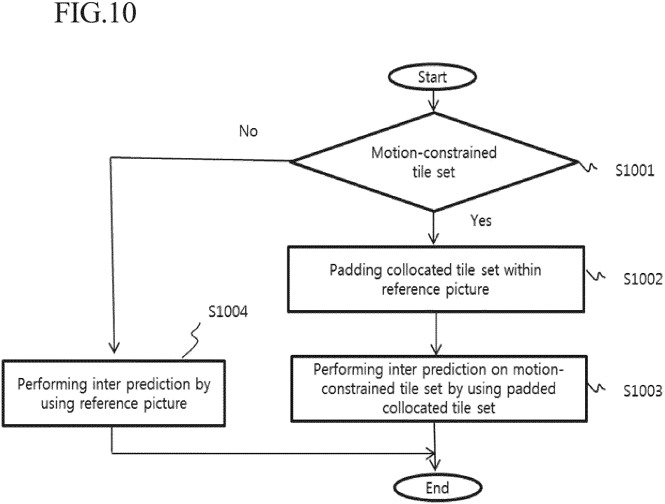

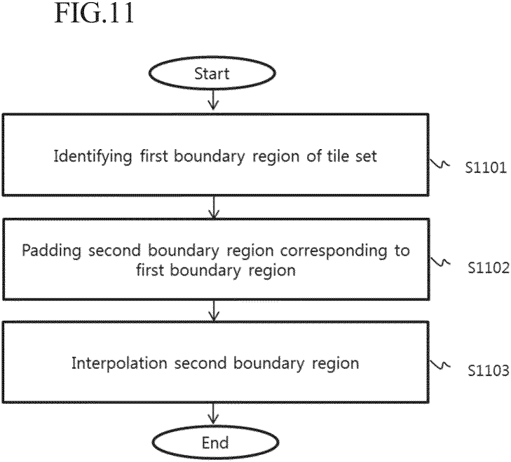

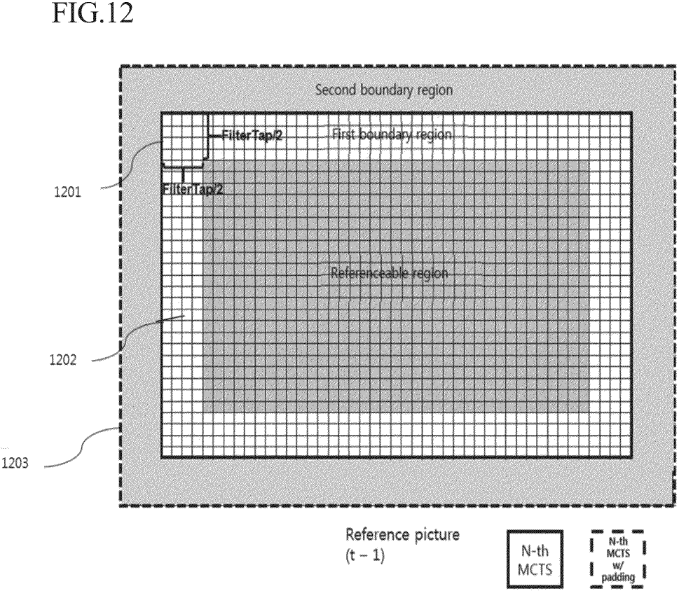

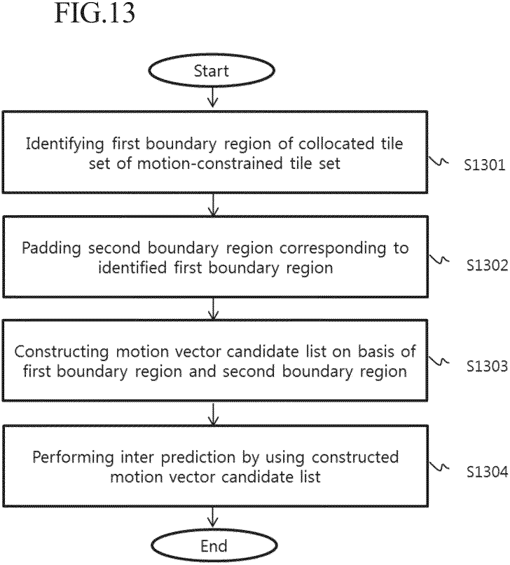

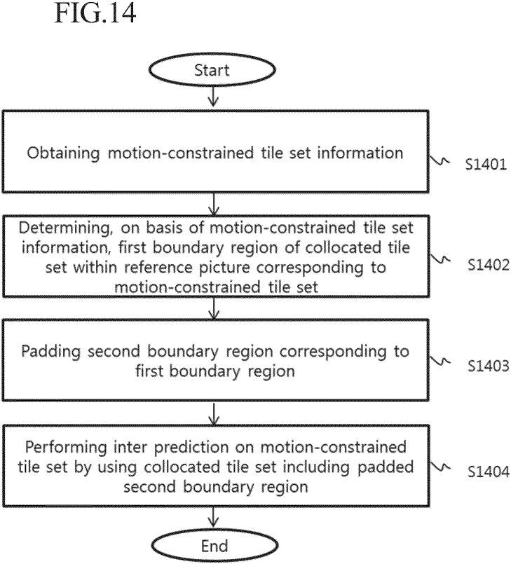

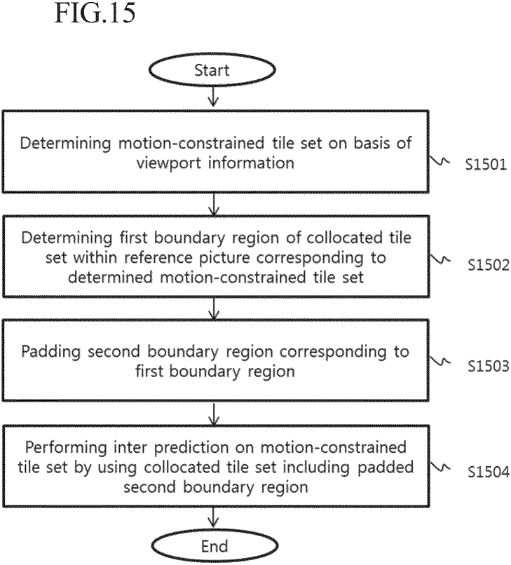

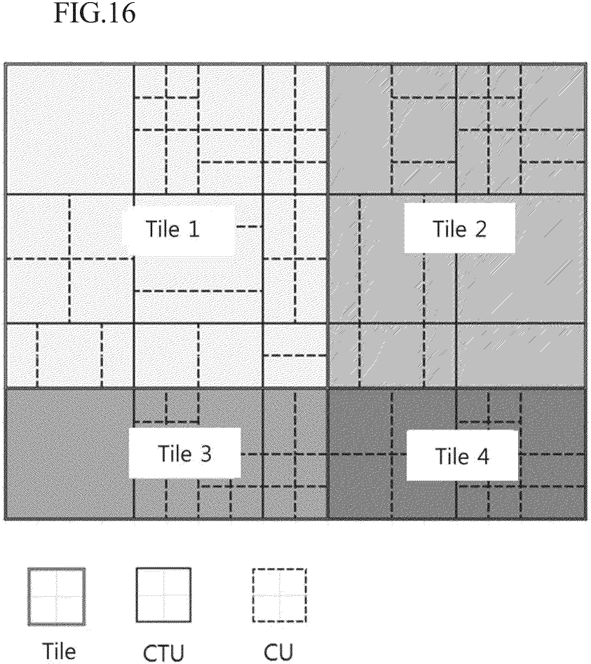

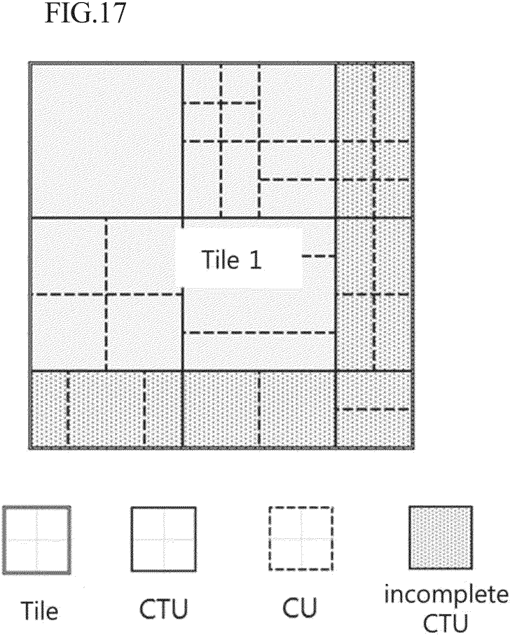

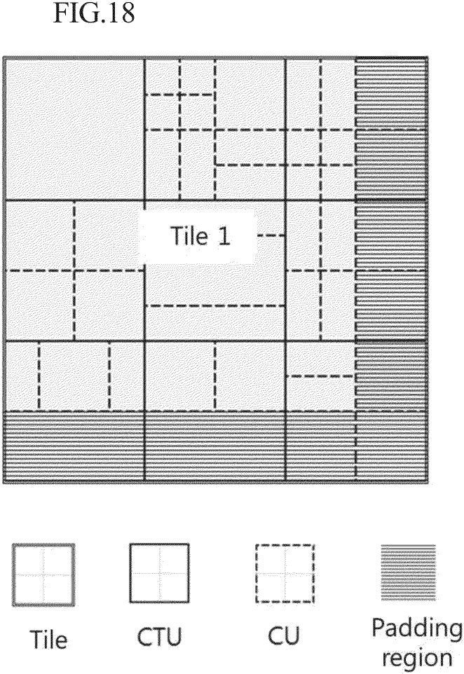

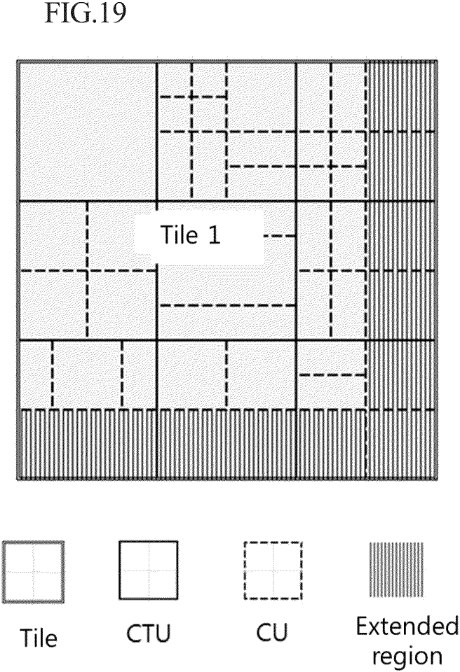

With reference to FIGS. 3E and 4, drive unit 300 may include processor 402, memory 404, and drive unit 402. Encoder disk 334 and encoder sensor 336 may determine the angular position, speed, and/or direction of the rotor and provide such information as positional feedback signal(s) to a processor 402 and/or memory 404. In other embodiments, a linear position sensor (not depicted) may be used to determine the position of the ball screw 326 or bellows 320 and to provide such positional feedback signal(s). Processor 402 and/or memory 404 may also receive control information from drive unit Ul 406 and provide display information (e.g., status information) to drive unit Ul 406. Relatedly, processor 402 and/or memory 404 may similarly receive EKG signal(s) from skin interface device 190 and one or more sensors 160 (FIG. 1), one or more external control signals (e.g., from a tablet or other computing device (not depicted)), and one or more sensor signals. For example, processor 402 and/or memory 404 may receive a pressure signal as observed by a pressure sensor (not depicted) located in proximity to balloon 110 when disposed in an artery (e.g., the descending aorta, as depicted in FIG. 1) of a patient undergoing therapy (e.g., counterpulsation therapy). The one or more pressure signals may be, indicative of the pressure exerted on balloon 110 in such artery and/or the pressure within such artery.

Processor 402 may use one or more of the positional feedback signals, drive unit Ul 406 control signals, EKG signals, external control signals, and sensor signals to control motor. For example, EKG signals may be used to ensure proper timing of the inflation and/or deflation of the balloon 110 (e.g., to pursue counterpulsation). And drive unit Ul 406 control signal and external control signals may be used to change the volume displacement and/or support ratio, as discussed supra.

Assuming drive unit 200 and drive unit 300 are configured to move the same amount of air when the corresponding bellows 220, 320 are subjected to linear motion from a fully expanded to a fully contracted position, or vice versa (i.e., to deflate or inflate the same size balloon), drive unit 300 may have several advantages over drive unit 200. For example, drive unit 300 can be configured to be smaller and lighter (i.e., have a smaller volume enclosure) than drive unit 200, which can have a significant impact on the ability of a person having to carry drive unit 150 to be mobile/engage in more ambulatory behavior. In particular, bellows 320 can have a smaller bellows travel distance (and a shorter ball screw) than drive unit 220. By increasing the bellows diameter in drive unit 300 relative to the bellows diameter in drive unit 200, (1) the impedance of the pneumatic load on the drive unit (e.g., the collectively pneumatic load of balloon 110, first pneumatic driveline 120, second pneumatic driveline 140, and external driveline 172) is better matched to the impedance of the motor (i.e., the mechanical subsystem of drive unit), and (2) the power consumption necessary to inflate and deflate is reduced, thereby increasing efficiency of drive unit 300 as compared to drive unit 200.

With such a configuration the overall dimension of drive unit 300 along its axis of linear motion (i.e., axis D-D) can be smaller than the overall dimension of drive unit 200 along its axis of linear motion (i.e., axis B-B). Whereas the longest dimension of drive unit 200 may be the along its axis of linear motion (i.e., axis B-B), the longest dimension of drive unit 300 may be orthogonal to its axis of linear motion (i.e., orthogonal to axis D-D). This may be particularly true where outer cases 202 and 302 are both a cuboid (or substantially in the shape of a cuboid) and are configured such that the face of static flange 229, 329 is parallel to a face of outer case 202, 302. As depicted in FIG. 3E, the longest dimension of drive unit 300 may correspond to an axis that is parallel to the diameter of bellows 320.

Further, the design of drive unit 300 may be configured to operate fast enough to deflate a corresponding balloon or inflatable member 110 by 50% within approximately 100 ms of the R-wave in the QRS complex, so that it may be capable of treating patients with irregular heartbeats (e.g., patients with atrial fibrillation or “a-fib”). As noted above, a-fib is an irregular and/or rapid heart rate that occurs when the two upper chambers of the heart experience chaotic electrical signals. The result is a fast and irregular heart rhythm that is difficult to predict for effective inflation and deflation of the expandable member 110. To effectively treat a-fib patients, the expandable member 110 should be deflated by at least 50% within approximately 100 ms of the R wave. Without rapid deflation, the expandable member 110 might obstruct blood flow within the aorta and increase the workload of the heart. Using the R-wave in a QRS complex as an indicator of ventricle contraction requires processing of an EKG signal to locate the R-wave, which can consume about 20-50 ms, depending on the algorithm employed using conventional algorithms and processors. By setting the ball screw pitch in drive unit 300 between 3.5 and 5.5 mm, and using a bellows 320 with an outside diameter between 125 and 111 mm with an effective bellow travel distance between 8.0 and 11.5 mm, drive unit 300 may be capable of effectively treating patients with irregular heartbeats and a-fib.

Even further, drive unit 300 can be far more efficient than drive unit 200 in terms of power consumption thereby prolonging battery life of the battery (not depicted) powering motor 322 as compared to battery (not depicted) powering motor 222, assuming the same such batteries are the same. In particular, motor 222 may inefficiently use a disproportional amount of power to overcome the rotational inertia of the rotor 222a in drive unit 200 as compared to the amount of power utilized in drive unit 300 to overcome the rotational inertia of rotor 322a. Drive unit 300 realizes this advantage by (a) setting the ball screw pitch in drive unit 300 such that the impedance of the motor 322 is the same or substantially the same (i.e., matched or substantially matched) to the impedance of the pneumatic load on the drive unit 300, and/or (b) using an enlarged bellows 320, which permits a design with a reduced bellows travel (and which may further help match or substantially match impedance of the motor 322 to the pneumatic load on the drive unit 300).

The above-provided tables for drive unit 200 and drive unit 300 are merely exemplary. Drive units with different component values are contemplated as being within the scope of this disclosure.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. The various embodiments described herein may also be combined to provide further embodiments.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and the combination of A and B. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly depicted or described herein.

What is claimed is:

1. A drive unit for controlling an expandable member in an intravascular circulatory support system, the drive unit comprising:
a motor including a rotor and a stator;
a ball nut carrying a ball screw, wherein the ball nut is affixed to the rotor; and
a bellows having a first end and a second end and a bellows cavity located between said first end and said second end, wherein:
the first end is defined as being fixed and as having a pneumatic output, and
the second end is defined by a dynamic flange configured to receive the ball screw, the dynamic flange having a recess directed toward the bellows cavity, the recess receiving and nesting at least a portion of the motor within the bellows cavity,
wherein the ball nut converts rotation of the rotor into linear motion of the ball screw within the ball nut and actuates the bellows, thereby moving air into or out of the pneumatic output.

2. The drive unit of claim 1, wherein:
the motor further includes a motor housing, and
the stator is fixed to the motor housing.

3. The drive unit of claim 1, wherein:
the ball screw includes a helical raceway,
the ball nut includes a plurality of balls that ride within the helical raceway, such that rotation of the rotor causes the plurality of balls to translate such rotation into linear motion of the ball screw within the ball nut.

4. The drive unit of claim 1, wherein the dynamic flange is fixed to the ball screw.

5. The drive unit of claim 4, further comprising an outer case having an inside surface wherein:
the first end is defined by a static flange that is fixed to an inside surface of the outer case,
the static flange seals the first end,
the dynamic flange seals the second end, and
the static flange includes a bellows outlet.

6. The drive unit of claim 1, wherein the shape of the recess is cylindrical.

7. The drive unit of claim 1, further comprising an encoder disk and an encoder sensor configured to:
determine one or more of the angular position, speed, and direction of the rotor; and
generate one or more positional feedback signals based on the determined one or more of the angular position, speed, and direction of the rotor.

8. The drive unit of claim 7, further comprising a processor configured to control the motor based at least on the one or more positional feedback signals.

9. The drive unit of claim 8, wherein the processor is further configured to control the motor based on one or more of EKG signals, wherein the one or more EKG signals are of a patient undergoing therapy and one or more pressure signals associated with the pressure within an artery of the patient.

10. The drive unit of claim 1, further comprising a processor configured to control the motor based on one or more EKG signals, wherein the one or more EKG signals are of a patient undergoing therapy.

11. The drive unit of claim 2, further comprising at least one radial bearings having an inner race and an outer race, wherein the outer race of the at least one radial bearings is affixed to the motor housing and the inner race of the at least radial bearings is fixed to the rotor-ball-nut assembly.

12. The drive unit of claim 1, wherein the ball screw is hollow.

13. The drive unit of claim 10, wherein:
the bellows has an outside diameter,
the outside diameter of the bellows is selected such that the drive unit can deflate the inflatable member by 50% within approximately 100 ms of receipt of an R-wave in a QRS complex detected in one or more EKG signals, wherein the one or more EKG signals are of a patient undergoing therapy.

14. The drive unit of claim 1, wherein the outside diameter of the bellows is within the range of approximately 111 mm and 125 mm.

15. The drive unit of claim 1, wherein the bellows travel distance is based on the selected outside diameter of the bellows.

16. The drive unit of claim 1, wherein the bellows travel distance is within the range of approximately 8 mm and 11.5 mm.

17. The drive unit of claim 1, wherein the length of an outer diameter of the bellows is approximately ten times as long as the bellows travel distance.

18. The drive unit of claim 3, wherein the pitch of the helical raceway is selected such that the impedance of the motor is the same or substantially the same as the impedance of the pneumatic load on the drive unit.

19. The drive unit of claim 1, wherein the diameter of the bellows is selected such that the impedance of the motor is the same or substantially the same as the impedance of the pneumatic load on the drive unit.

20. The drive unit of claim 16, wherein the pitch of the helical raceway is within the range of approximately 3.5 mm and 5.5 mm.
